(19) United States
(12) Patent Application Publication
(10) Pub. No.: US 2017/0232253 A1
(43) Pub. Date: Aug. 17, 2017

(54) Bioelectrodynamics Modulation Method

(71) Applicants: Marcy C. Purnell, Hernando, MS (US); Michael A. Whitt, Cordova, TN (US)

(72) Inventors: Marcy C. Purnell, Hernando, MS (US); Michael A. Whitt, Cordova, TN (US)

(21) Appl. No.: 15/044,972

(22) Filed: Feb. 16, 2016

(51) Int. Cl. A61N 1/20; A61N 1/04; A61K 33/14

(52) U.S. Cl. CPC A61N 1/20; A61K 33/14; A61N 1/0472

(57) ABSTRACT

Disclosed are methods and devices utilizing bioelectrodynamics for the therapeutic treatment of disorders in a subject in need thereof with an electric field or electromagnetic field therapy. The methods and devices provide an inhibitory effect on growth of cancer cells that are exposed to an electromagnetic field generated by a CETS unit. Cancer cells may arrest proliferation and enter apoptosis by activation of the unfolded protein response (UPR), TNF/TRAIL, and p53 oncogene activation. The methods and devices may be used to enhance wound healing with exposure to an electromagnetic field generated by a CETS unit. Noncancerous cells may show a significant increase in cell migration with no activation of apoptosis pathways.

### FIELD OF THE INVENTION

The presently disclosed subject matter relates to a method and devices for the therapeutic treatment of disorders in a subject with electric field or electromagnetic field therapy.

### BACKGROUND OF THE INVENTION

The WHO's World Cancer Report of 2014 has stated that cancer will become a significantly larger global health care burden in the next two decades with the cancer incidence expected to increase by 57% and deaths are projected to increase by 63% (Wild, 2014). Secondly, while cancer survival rates are increasing in our society, a direct consequence of extended survivorship is the development of secondary primary malignancies as well as ongoing side effects of the present day toxic cancer treatment (Kalaycio, 2014). For years, researchers have worked diligently to find the coveted treatment that would target cancer cells without harming healthy cells. This goal continues to remain elusive. Recent research has suggested that cancer may be an actual developmental disorder and may occur when cells stop responding to normal patterning cues of the body (Lobikin, Chernet, Lobo, & Levin, 2012). Cancer could be described as an inexorable process where the organism fails to maintain its efforts to maintain order (Lobikin et al., 2012). This view of cancer as a reversible physiological state has potentially significant medical implications. If it would be possible to modulate or impact the cellular environment so as to affect progression to neoplasms, this could change how we detect, treat and prevent cancer. Today therapies are limited to killing tumors and this approach unfortunately leads to damaging the healthy cells and often the individual in the process.

The global wound care market is expected to reach $18.3 billion by 2019. The ever increasing aging and diabetic populations will continue to contribute to these costs. It is believed that new product development and innovation will be leading players in the wound care market. New treatments that speed healing and/or reduce infection would make a significant impact on overall health and reduce medical care costs.

Hypertension is a chronic disease that is often under-estimated as an economic burden while incurring the highest drug expenditures in the United States. Current pharmaceutical therapy can be cost prohibitive with unwanted side effects. New strategies that have the potential to lower blood pressure would make a lasting and significant impact on the health of the U.S. population, particularly in view of the increase incidence of obesity and the link to hypertension and cardiovascular disease.

### SUMMARY OF THE INVENTION

In one aspect, the present invention provides a method of the therapeutic treatment of a disorder in a subject, comprising applying an electric field that inhibits the growth of the cancer cells, but leaves normal cells substantially unharmed, wherein the subject or a portion of the subject is immersed in a therapeutically effective amount of a composition under the electric field. In some embodiments, the composition is in solution. In some preferred embodiments, the solution comprises a salt solution, such as a sodium chloride solution, at concentration of about 2 mM to about 5 mM. In some embodiments, the composition comprises an electro-activated solution. In preferred embodiments, the composition is subjected to an electric field of between about 100 mV and 900 mV and up to 2.5 amperes of direct current. In more preferred embodiments the composition is subjected to an electric field of 130 mV and 2.5 amperes of direct current. The electric field may be generated using a series of spaced rings. In other preferred embodiments the composition is subjected to an electric field using a cellular energy transfer system (CETS). The composition may be subjected to an electric field for a duration of up to about 35 minutes. The method may be used to inhibit the growth of cancer cells in the subject.

In another aspect, the present invention provides a method for the therapeutic treatment of a disorder comprising subjecting a therapeutic agent to an electric field; and applying a therapeutically effective amount of the therapeutic agent to a subject in need of treatment therefrom, to thereby limit the occurrence of cancer or treat cancer in the subject. In some embodiments, subjecting a therapeutic agent to an electric field comprises subjecting the therapeutic agent to an electric field of between about 100 and about 900 mV and up to about 2.5 amperes of direct current. In preferred embodiments, subjecting a therapeutic agent to an electric field comprises subjecting the therapeutic agent to an electric field of 130 mV and 2.5 amperes of direct current. Subjecting a therapeutic agent to an electric field may comprise generating an electric field using a series of spaced rings. In other preferred embodiments the electric field is generated using a cellular energy transfer system (CETS). In some embodiments, subjecting a therapeutic agent to an electric field may comprise subjecting a therapeutic agent to an electric field for a duration of up to about 35 minutes. The therapeutic treatment results in inhibition of growth of cancer cells.

In still another aspect, the present invention provides a composition prepared by a process comprising the steps of providing a composition comprising a conductive agent, provide an electric field of between about 100 mV and about 900 mV and up to about 2.5 amperes of direct current, and subjecting the composition under the electric field for a duration of up to 35 minutes. In some embodiments, the composition is an electro-activated solution. In some preferred embodiments, the electro-activated solution is a salt solution, such as a sodium chloride solution, at concentration of about 2 mM to about 5 mM. In more preferred embodiments the composition is subjected to an electric field of 130 mV and 2.5 amperes of direct current. The electric field may be generated using a series of spaced rings. In other preferred embodiments the composition is subjected to an electric field using a cellular energy transfer system (CETS). The composition may be used to inhibit the growth of cancer cells in a subject.

### BRIEF DESCRIPTION OF THE DRAWINGS

Further advantages of the invention will become apparent by reference to the detailed description of preferred embodiments when considered in conjunction with the drawings which form a portion of the disclosure and wherein:

FIG. 1 is a bar graph illustrating the growth of B16 mouse melanoma cells in treated and untreated media.

FIG. 2 is a bar graph showing the growth of non-cancerous mouse L929 fibroblasts in treated and untreated media.

FIG. 3 is a bar graph showing the growth of human breast cancer cells (MDA-MB-231) in treated and untreated media.

FIG. 4 is a bar graph showing the growth of normal human breast epithelial cells (MCF-10A) in treated and untreated media in a wound healing assay.

FIG. 5 is a graph showing the differential response of mouse L929 fibroblast cells in treated and untreated media in a wound healing assay.

FIGS. 6A & 6B show microarray heat maps. FIG. 6A shows an Affymetrix 2.0 gene expression values (which are provided in detail within TABLE 2) for ER stress/UPR, immune/TNF, cell cycle, tumor targets, cell death and membrane potential across the four conditions of the control and treated of the MDA-MB231 and MCF-10A cell lines. FIG. 6B shows expressions of genes validated in real time PCR across the four conditions of the control and treated MDA-MB231 and MCF-10A cell lines.

FIG. 7 shows real time RT-PCR experiment validations with gene expression fold change by the double delta CT method in MDA-MB231 cell lines.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. Further, while the terms used herein are believed to be well-understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently disclosed subject matter relates to a method and devices for the therapeutic treatment of disorders in a subject with electric current or electromagnetic field therapy. More particularly, the presently disclosed subject matter relates to a method of applying an electric field that inhibit the growth of the cancer cells, but leaves normal cells substantially unharmed. In some embodiments, the subject or a portion of the subject is immersed in a therapeutically effective composition under the electric field. Non-limiting examples of the disorder are cancer and wounds. In some embodiments, non-limiting examples of cancer are breast cancer and melanoma. In some embodiments, the composition is an aqueous solution. In some embodiments, the composition is an electro-activated solution. In some embodiments, the composition comprises a conductive agent. Non-limiting example of the conductive agent is sodium chloride, but other water soluble salts known in the art may be used.

In some embodiments, the presently disclosed subject matter provides a method of treating a disorder in a subject. The method includes the steps of immersing the subject or portion of the subject to a composition, and providing an electric field to the composition to treat the disorder. In some embodiments, the composition is in solution. In some embodiments, the composition comprises conductive agents. In some embodiments, the composition is an aqueous electro-activated solution. In some embodiments, the aqueous solution includes a salt solution, such as a sodium chloride solution.

In some embodiments, the present application relates to a method of administering a therapeutically effective amount of a composition that was previously subjected to an electric field, thereby limiting the occurrence of the disorder or treating the disorder in the subject. In some embodiments, the composition is in solution. In some embodiments, the composition comprises conductive agents. In some embodiments, the compositions are an aqueous electro-activated solution. In some embodiments, the aqueous solution includes a salt solution, such as a sodium chloride solution. In some embodiments, the composition was previously subjected to an electric field.

In some embodiments, the electric field is generated using a series of spaced rings. In some embodiments, the electric field is generated by a direct current. In one embodiment, the composition is subjected to an electric field of between about 100 mV and 900 mV and up to 2.5 amperes of direct current. In some embodiments, the electric field is between about 100 mV and about 900 mV. The electric field is about 100 mV, 150 mV, 200 mV, 250 mV, 300 mV, 350 mV, 400 mV, 450 mV, 500 mV, 550 mV, 600 mV, 650 mV, 700 mV, 750 mV, 800 mV, 850 mV, and 900 mV. In some embodiment, the direct current is less than or equal to about 2.5 amperes. The direct current is about 0.5 amperes, 1 amperes, 1.5 amperes, 2.0 amperes, and 2.5 amperes. In one embodiment, the composition is subjected to an electric field of 130 m V and 2.5 amperes of a direct current. In some embodiments the direct current is supplied by a voltage source of less than about 220 volts. In some embodiments, the direct current is pulsed. In some embodiments, the subject is treated with the direct current for a time period of less than about 35 minutes. In some embodiments, the subject is treated with the direct current for about 5 minutes, 10 minutes, 15 minutes, 20 minutes, 25 minutes, 30 minutes, and about 35 minutes.

In some embodiments, the presently disclosed subject matter relates to a device for treating a disorder in a subject. The device includes a reservoir, an aqueous solution in the reservoir for contacting the subject or a portion of the subject, a first electrode in the reservoir, a second electrode in the reservoir, and an apparatus for providing an electric field or electromagnetic field to the aqueous solution to treat the disorder. In some embodiments, the electric field is generated using a series of spaced rings. In some embodiments, the electric field is generated by a water treatment apparatus as disclosed in U.S. Pat. No. 6,555,071, which is incorporated by reference in its entirety. In some embodiments, the aqueous solutions were previously subjected to an electric field. In some embodiments, the subject or a portion of the subject is immersed in the aqueous solution under an electric field. In some embodiments, the aqueous solution comprises a conductive agent.

In some embodiments, the presently disclosed subject matter provides a composition comprised of a conductive agent. Generally, any conductive agent that can generate an electric field in the ranges of about 100 mV to about 900 mV and up to about 2.5 amperes can be used. In some embodiments, the composition is in solution. Non-limiting examples of the solution include tap water, bore water, distilled water, and salt solutions, such as a sodium chloride solutions. In some embodiments, the salt solution, such as a sodium chloride solution, is in a concentration of about 2 mM to about 5 mM. The salt solution, such as a sodium chloride solution, is in a concentration of about 2 mM, 2.5 mM, 3.0 mM, 3.5 mM, 4.0 mM, 4.5 mM, and 5.0 mM.

In some embodiments, the presently disclosed subject matter provides a therapeutic treatment resulting in the inhibition of growth of cancer cells. In some embodiments, the therapeutic treatment resulting in apoptosis of cancer cells.

In one embodiment of the present application, the therapeutically effective composition is subjected to an electric field generated using a series of spaced rings. In some embodiments, the electric field is generated by a cellular energy transfer system (CETS). In some embodiments, the device Cellular Energy Transfer System (CETS) unit generates an electric field with 130 mV and 2.5 amperes of direct current (DC) through a series of stainless steel and copper rings when placed in a 3 mM hypotonic saline solution. The CETS unit has been used across the globe for several decades with positive effects on the health of living organisms such as plants, animals and humans (Marsh, 2001; Walker, 1998, hereby incorporated by reference in its entirety). Reports of these positive effects, which range from anecdotal testimonials by individuals using the CETS unit in a footbath to semi-controlled studies published in the literature, have led us to test the validity of these claims through rigorous scientific experimentation. A review of the literature has shown that it has been recognized that the electrical potential across the membranes of cancerous cells (−30 mV) differs compared to noncancerous cells (−70 mV) and that a more positive potential correlates with increased mitotic activity (Cone, 1970). Recently it was also suggested that modulating the membrane potential of cancer cells may be used to inhibit tumor growth (Chernet & Levin, 2013; Yang & Brackenbury, 2013). There are actually three types of cells that have been found to have a low transmembrane potential: cancerous, injured and proliferating cells (Yang & Brackenbury, 2013). With the reports that were observed with the CETS unit and the documented differences in the transmembrane potential between cancerous and noncancerous cells, as well as the low transmembrane potential in injured and proliferating cells, the presently disclosed subject matter provides experiments to test these observed phenomena with cancerous and noncancerous cell lines in vitro.

The term “bioelectrodynamics” refers to the “critical roles of electromagnetism and mechanics in biology” in a living organism, correlation of “biophysical functions of living organisms with biochemical processes at the cellular level,” and introducing “theoretical basis and methodology, such as modeling and simulations, for stimulating technical innovations and promoting technology development in biomedicine as well as for the study of human healthcare issues related to environments . . . ” (Zhou & Uesaka, 2006). Bioelectricity (electromagnetism in organisms) refers to the changing gradients of transmembrane potential, ion fluxes, cell signaling pathways and electric fields produced and sensed by excitable and non-excitable cells (Levin, 2007). Due to recent development of molecular resolution tools, it is now known that transmembrane (Vm) voltage gradients control cell proliferation, migration, differentiation and orientation (Sundelacruz, Levin, & Kaplan, 2009). The recent research in the area of bioelectrodynamics all point to bioelectricity as being a non-genetic aspect of the cancer microenvironment (Lobikin et al., 2012). The view that cancer is a developmental disorder may predict that molecular mechanisms can be involved in tumorigenesis. While researchers have argued that cancerous transformations occur due to changes in the genetic code such as mutations of p53 and BRCA1 tumor suppressor genes, there is also now mounting evidence that bioelectric cues can go awry and result in cancerous growth (Lobikin et al., 2012; Mijovic et al., 2013). As stated earlier, cancer cells are generally depolarized with respect to healthy tissue (Cone, 1970). A number of ion channels as well as pump and gap junction genes are also now recognized as actual oncogenes. Epigenetic chromatin and histone modifications in the nucleoplasm (acetylation/de-acetylation) have also been shown to be affected by bioelectric cues (Tseng & Levin, 2013). Electrical oscillations of the right frequency and amplitude can alter electrical charge distribution in the cell receptors leading to conformational changes in the cell just as if activated by a chemical signal (Charman, 1996). Additionally, recent studies describe how electrical potential, or manipulation of membrane potential, can affect cells. (Movaffaghi & Farsi, 2009; Kevin W. Chen, 2008; Huang et al., 2013; Ojeda-May & Garcia, 2010; Funk et al., 2008; Mayer, 2014; Hu & Verkman, 2006; Ohnishi et al., 2005; Panagopoulos, et al., 2002; Yang and Brackenbury, 2013; Romanel et al., 2012; Chernet and Levin, 2013; Gregory et al., 1997).

In some embodiments, the presently disclosed subject matter provides an electromagnetic field produced by the CETS unit confers a change to either the ions in the dilute saline solution, or the water itself, which then modulates the membrane potential of cells. In some embodiments, the effects may result from changes in the activity of various ion channels (such as Na+ channels, K+ channels, Ca++ channels, etc.), or water channels (e.g. aquaporins), in the cell membranes which would subsequently affect the charge differential across the membranes.

Further provided, in some embodiments of the presently disclosed subject matter, is a method for the therapeutic treatment of a disorder. The method includes subjecting a therapeutic solution to an electric field, and applying a therapeutically effective amount of the therapeutic solution to a subject in need of treatment therefrom, to thereby limit the occurrence of cancer or treat cancer in the subject. In some embodiments, the subject is treated by immersing the subject or a portion of the subject, such as the feet, in the therapeutic solution during, or after the exposure of the solution to the electric field. In some embodiments, the therapeutic solution is treated with an electric field before immersing the subject, or a portion of the subject in the solution. In some embodiments, the therapeutic solution is subjected to an electric field during the exposure of the subject. In some embodiments, subjecting a therapeutic solution to an electric field comprises subjecting the therapeutic agent to an electric field of between about 100 and about 900 mV and up to about 2.5 amperes of direct current. In one embodiments, subjecting a therapeutic solution to an electric field comprises subjecting the therapeutic solution to an electric field of 130 mV and 2.5 amperes of direct current. In one embodiment, the electric field is generated using a cellular energy transfer system (CETS). In some embodiments, the solution is an electro-activated solution. In some embodiments, subjecting a therapeutic agent to an electric field comprises subjecting a therapeutic agent to an electric field for a duration of up to about 35 minutes. In some embodiments, the therapeutic treatment results in the inhibition of growth of cancer cells. In some embodiments, subjecting a therapeutic agent to an electric field comprises generating an electric field using a series of spaced rings.

Still further provided in some embodiments of the present disclosure, is a composition prepared by a process. The process include the steps of providing a composition comprising a conductive agent, provide an electric field of between about 100 mV and about 900 mV and up to about 2.5 amperes of direct current, and subject the composition under the electric field for a duration of up to 35 minutes. In some embodiments, the composition is an electro-active solution. In some embodiments, the composition is a saline solution in a concentration of about 2 mM to about 5 mM.

In some embodiments of the presently disclosed subject matter provides a method of producing the composition comprising the steps of providing a composition, wherein the composition comprising a conductive agent, providing an electric field of between about 100 mV and about 900 mV and up to about 2.5 amperes of direct current, and subject the composition under the electric field for a duration of up to about 35 minutes.

In some embodiments, the composition treated by an electric field can be used to treat a variety of health conditions, including, but not limited to; cancer, hypertension, wound care/healing, asthma, COPD, autism, ADHD, insomnia, renal dysfunction, liver dysfunction, fluid retention, immune system enhancement, arthritis, diabetes, clotting disorders, psoriasis, eczema and acne. In some embodiments, when properly scaled-up, there can be benefits to horticulture applications including, but not limited to; increased crop yield, reduced need for watering, and overall plant health. In some embodiments, the presently disclosed subject matter provides that tumor cells maintained in media prepared with CETS-treated water have a much reduced rate of growth whereas non-tumorigenic cells grown in the same media are not affected. In some embodiments, the use of the CETS unit can lower blood pressure of individuals with hypertension.

The presently-disclosed subject matter is further illustrated by the following specific but non-limiting examples. Some of the following examples are prophetic, notwithstanding the numerical values, results and/or data referred to and contained in the examples.

### EXAMPLES

### Example 1

This study provides data to support the use of the CETS unit as a medical device to treat hypertension. In this study, a controlled human trial is designed to test whether individuals with elevated blood pressure (defined as men or women, ages 35-65 of any race who have prehypertension or stage I or II hypertension) show a sustained reduction in blood pressure after use of the device.

There have been several studies in this country and abroad examining the effect of the CETS unit on hypertension. One pilot study based on 12 treated subjects showed a drop in blood pressure of 4.1 (+/−3.3) mm Hg for systolic blood pressure and 8 (+/−6.5) mm Hg for diastolic. While this rivals results obtained with present day pharmaceuticals that are used to manage blood pressure, there was not a control arm in this study and the results have not been published in a peer-reviewed journal.

To determine if the CETS unit can indeed lower blood pressure, a protocol is designed for a clinical trial. The pilot trials and the current utilization of the CETS unit consist of individuals placing their feet in a footbath every other day for thirty minutes with the CETS unit running so that 130 mV and 2.5 amperes is delivered to the dilute saline solution in the bath. The double-blind, randomized, controlled study includes 20 pre-hypertensive or hypertensive participants who qualify for the trial through the specified inclusion/exclusion criteria. Both treatment and control groups are subjected to the same physical environment of sitting in a comfortable chair with their feet submerged in the footbath together with the CETS unit. For the control group, the power pack is turned on, but the electrodes are not plugged into the unit. The participants are not able to see the footbath as it is hidden beneath a table with a cloth extending down to the floor. This ensures the participants cannot discern whether the unit is operating or not since some small bubbles are produced when the unit is connected to the power pack and current is being delivered to the unit.

The subjects have their blood pressures monitored at each and every visit to assess the safety and efficacy of the treatments. They will also have historical data collected by the research coordinators. In the event there is any concern about the potential effects of the treatments with the CETS unit, an evaluation occurs immediately by the principal investigator and the subject is referred to the appropriate medical personnel. These potential effects could be blood pressure elevated above 160/90 or below 100/50 as well as any other concerns.

Exclusion criteria include anyone in end stage renal disease, individuals who have had an organ transplant, anyone diagnosed with cancer within the last 5 years, as well as individuals who have cardiovascular disease, congestive heart failure, or women who are pregnant (women of child bearing age must be have a pregnancy test prior to entering the trial). Individuals diagnosed with psychiatric disorders, have more than 21 alcoholic drinks a week, or those who use illicit drugs will also be excluded from the study.

The subjects have blood pressure measurements taken at their screening visit to verify they have hypertension. If they meet the requirements for eligibility by inclusion/exclusion criteria, they will be brought in for a randomization visit. They then have a physical exam prior to randomization. If the physical exam is satisfactory and they have qualified for the trial, they will be randomized to either the control or treated group.

Treatment adherence and study compliance will be assessed by the subjects showing up for each and every clinic visit to receive the footbath treatments with the CETS unit. Subjects will be withdrawn from the study if they miss more than 10 percent of the required study visits since this could adversely affect the results. If this occurs the subjects will be brought in for their final visit and appropriately and safely closed out of the study. The subjects will be replaced by recruiting new subjects in order to achieve 20 subjects completing the study.

The primary endpoint of the study is the development of malignant hypertension or a blood pressure of greater than 170/100. The secondary endpoint will be a hypotensive episode or a symptomatic blood pressure of less than 100/50. Blood pressure measurements will be taken prior to each footbath and after completion of each footbath. The average of three blood pressure measurements taken 1 minute apart will be recorded. In the event a primary or secondary endpoint is found upon these measurements, the appropriate actions will be taken.

The primary objective of the study is to compare changes in blood pressure of the treatment group to the control group over time. The secondary objective is to compare quality of life measurements between the two groups. The participants in the treatment group will receive 30 minute footbaths three times a week with the CETS unit “turned-on” for one month. The participants in the control groups will receive 30 minute footbaths three times a week without the CETS device turned-on. Blood pressure measurements will be taken at every visit prior to and after the administration of the footbath. The quality of life questionnaire, PROMIS “Patient Reported Outcome Measurement System” from the National Institutes of Health, will be administered to the participants at the first visit (after randomization) and the final visit. The outcome variables are continuous variables. Hence a unified statistical method using mixed modeling will be conducted to compare the two groups on these outcome variables. The mixed model consists of the treatment, time, and treatment by time interaction effects. The effect of interest is the treatment by time interaction which will show unequal change in the outcome variables over time. SAS 9.3 (SAS Institute, Cary, N.C.) will be used for this analysis.

### Example 2

This example shows that that treatment with the CETS unit inhibits cancer cell, but not normal cell growth. In this study, the growth of cells in a media prepared with dilute 3 mM saline solution treated with the CETS unit is examined. It is hypothesized that there would be a differential effect on the growth of cancerous compared to non-cancerous cells when grown in the media prepared with a dilute saline solution that had been exposed to the electromagnetic field produced by the CETS unit.

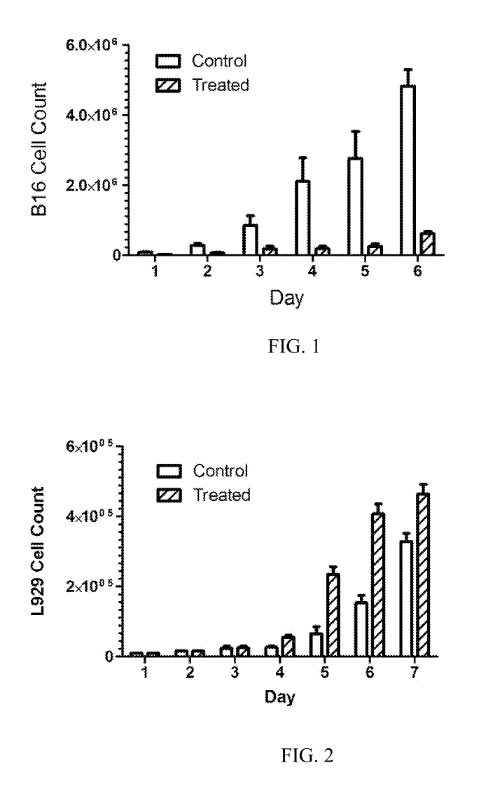

To test this hypothesis mouse melanoma (B16 cells) and normal mouse fibroblasts (L929 cells) are used to examine these effects on cell growth. B16 melanoma or normal L929 fibroblasts were cultured in Dulbecco's Modified Eagle's Medium (DMEM) that was reconstituted with a hypotonic saline solution (3 mM NaCl) that had been treated with the CETS unit (treatment groups) versus DMEM with prepared with the same saline solution prior to treatment with the CETS unit (control groups). Both treated and untreated media were supplemented with 10% Fetal Bovine Serum (FBS) after reconstitution. Aliquots of B16 or L929 cells were plated into culture dishes. The experiment was conducted over six to seven days and was repeated three to five times. Daily cell counts were performed in triplicate utilizing either a hemocytometer after staining with trypan blue to identify live versus dead cells in culture, or with an automated cell counter (Millipore Sceptor). Both treated and control media were prepared daily and the media was replaced each day until all counting was completed. Model fixed effects analyses were performed to determine if there were significant differences in growth between treatment groups using SAS 9.3. The melanoma B16 cells showed a significant reduction in mitosis when grown in the treated media as compared to B16 cells grown in untreated, control media starting on Day 4 (FIG. 1). In contrast, the noncancerous L929 fibroblasts (FIG. 2) showed no inhibition of growth in the treatment group when compared to the control group and instead it appeared there was a slight increase in the rate of growth, which we interpreted as an increase in the health of the cells.

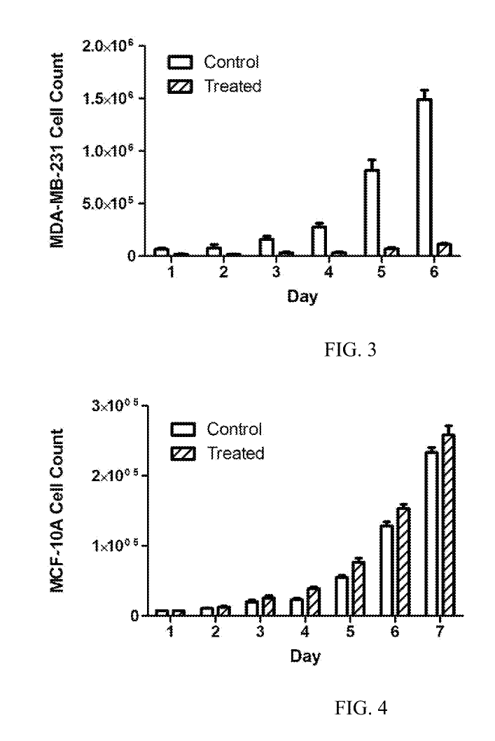

To determine if the growth inhibitory effects were seen in other cell types, the experiment is repeated using human MDA-MB-231 cells, which is a triple-negative breast cancer cell line and this was compared to MCF-10A cells, which are a normal breast epithelial cell line. Similar to the B16 melanoma cells, the MDA-MB-231 cells showed a significant reduction in mitosis (FIG. 3) when grown in the treated media as compared to the cells grown in the control media. Also similar to the normal murine L929 cells, the MCF-10A normal human breast line showed no inhibition of growth (FIG. 4) when grown in either the treated or control media. To investigate the mechanism(s) responsible for the significant growth inhibition of the cancerous cells and lack of growth inhibition of the noncancerous cells, cell cycle analysis by flow cytometry was performed. To examine these effects on cell cycle progression, cells were cultured in control or treated media as stated above. A third group was also cultured in Dulbecco's Modified Eagle's Medium with 10% FBS that was purchased, standard DMEM (e.g. was not reconstituted with the hypotonic saline solution). Aliquots of each cell type were plated into culture dishes. After three days exposure to the three different types of media (treated, controlled and standard DMEM), the cells were trypsinized, washed twice with PBS containing 0.1% FBS and then subsequently fixed in −20° C. ethanol. After overnight incubation at 4° C. the cells were washed twice in PBS and resuspended at a final concentration of 10⁶ cells/ml. The cells were then stained with propidium iodide in PBS and treated with DNase-free RNase A to remove remnants of RNA prior to cell cycle analysis by flow cytometry (TABLE 1).

TABLE 1
Cell Cycle Analysis

| Cell Line | G0-G1 Phase | S Phase | G2-M Phase |
|---|---|---|---|
| MDA-MB231-Treated | 72.61% | 25.27% | 2.12% |
| MDA-MB231- Control | 87.31% | 8.77% | 3.92% |
| MDA-MB231- DMEM | 86.52% | 10.87% | 2.62% |
| MCF-10A- Treated | 95.32% | 0.75% | 3.93% |
| MCF-10A- Control | 96.01% | 0.00% | 3.99% |
| MCF- 10A- MEMB | 93.34% | 2.94% | 3.72% |
| Bl6- Treated | 73.66% | 0.00% | 26.34% |
| Bl6- Control | 44.22% | 37.03% | 18.75% |
| Bl6-DMEM | 47.38% | 34.31% | 18.31% |
| L929- Treated | 68.46% | 31.54% | 0.00% |
| L929- Control | 71.23% | 28.28% | 0.50% |
| L929-DMEM | 62.71% | 37.29% | 0.00% |

The results indicated that the MDA-MB-231 cells grown in the treated media accumulated in the S phase of the cell cycle, suggesting they are unable to complete mitosis, but have increased DNA content. There was no apparent difference between the control group of MDA-MB-231 cells and the MDA-MB-231 cells grown in the purchased DMEM.

The mouse B16 melanoma cells grown in the treated medium, for comparison, showed a ˜2-fold increase of cells that accumulated in the G0-G1 phase with a complete reduction (0%) of cells in S phase. Thus there appears to be different effects on where the growth arrest is occurring between the murine melanoma and human breast cancer cell lines, which may be due to differences in the types of mutations that are driving the uncontrolled growth of a particular tumor type. Regardless of where the arrest is occurring, the effect is very clear, that growth in the treated media results in changes to the cell cycle, which are at least part of the molecular basis for the growth inhibition.

In contrast to the tumor cells, the normal human breast (MCF-10A) and normal mouse fibroblast (L929) cells showed no difference in progression through the cell cycle from the treated and control groups, which correlate to the cell growth studies shown in FIGS. 2 & 4. These results show that the cell growth studies shown in FIGS. 1-4 correlate with changes in cell cycle progression for the cancer cell group only, and only when those cells are maintained in the treated media.

In summary, it is found that there is an inhibitory effect on growth of two different cancer cell lines from two different species when grown in media prepared with “water” that had been exposed to the electromagnetic field generated by the CETS unit.

This device has been used worldwide in holistic markets since 1996 without any adverse side effects and the results of these studies indicate that there is a differential effect on cancer vs. noncancerous cell lines. This is the first time that anyone has examined the potential beneficial effects of the CETS unit towards cancer treatment. By identifying the molecular basis of the effects observed may offer a completely new avenue for treating patients with cancer, and while not a cure, it certainly could serve as an adjuvant to current therapies and reduce unwanted adverse side-effects that are common with most cancer treatments used today.

### Example 3

This study relates to wound healing effect of CETs unit treated solution using scratch assay with L929 cells.

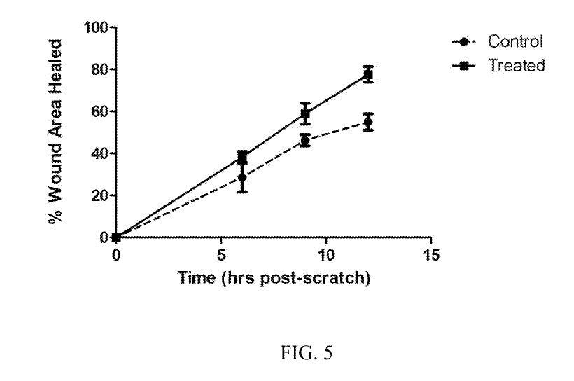

Mouse L929 fibroblast cells were plated in 6 cm dishes in DMEM with 10% FBS and allowed to reach ˜90% confluence on day 1. A marker line was placed on the bottom of each dish prior to plating. On day 2, using a sterile 200 microliter pipetteman tip, three separate wounds were made in the cell monolayer perpendicular to the line drawn on the bottom of the dish. The media in each of the 6 dishes was then aspirated and replaced with either CETS treated media (3 plates) or control (non-treated) media (3 plates). The treated media was DMEM with 10% FBS that had been made from a 10× solution and reconstituted with a 3 mM hypotonic saline solution that had been treated with the CETS unit. The control media was DMEM with 10% FBS that was made from a 10× solution that was reconstituted with the same 3 mM hypotonic saline solution taken immediately prior to treatment with the CETS unit. Phase-contrast images of the wounds in the monolayer were acquired immediately after addition of the treated or untreated media (time 0). The three wound areas were photographed on each plate with orientation to both the perpendicular line and the marker line noted above. Each picture was labeled for identification in order to compare the percent area change over time of each wound. The cultures were then place back in the 37° C., 7% CO₂ incubator. Cultures were removed from the incubator at 6 hours, 9 hours and 12 hours and each wound (n=18) was photographed at each time point. The area of each wound was then calculated using NIH Image-J software for each time point photographed: 0 hours, 6 hours, 9 hours and 12 hours. The percent change in the area of the wound that lacked cells (e.g. wound healing) was then calculated. Paired t-tests were conducted on the differences in the % area of each wound between the treated and control groups. The results are: mean=15.000; S.D.=12.0830; S.E.=4.0277; P=0.0058 (FIG. 5).

These results indicate there is a significant difference in the rate of migration of cells that had been cultured in the treated media compared to the rate of migration of cells grown in the control media. Since rate of migration into the wound area is a surrogate measure of wound healing, these data suggest that treatment with the CETS unit may promote faster wound healing in patients.

### Example 4

Microarray analyses: MDA-MB231 and MCF-10A (2×10⁶) treated and (5×10⁵) untreated cells were placed in 60 mm dishes. Five dishes were plated for the treated groups and five for the control groups and were placed in DMEM-10 medium on day 1. Day 2—DMEM-10 was replaced with either treated or control media and the media were replace again on days 3 and 4. On day 5, the cells were trypsinized in all 10 plates and removed from plates and counted with Scepter cell counter to determine cell viability and 3×10⁶ cells from each plate (5 treated and 5 control) were placed in 2 ml Eppendorf tubes pelleted at 500 rpm for 5 minutes, the supernatant removed and the cells were re-suspended in 350 mcl RLT buffer and allowed to sit for 20 minutes on ice prior to being placed in microcentrifuge and spun for 3 minutes at 1,000 rpm. The supernatant was transferred to new 2 ml Eppendorf tubes. RNA isolation was conducted with the QIAcube using 70% ethanol, buffer RWI, buffer RPE, RNase free water and treated with DNase 1 according to the manufacturer's instructions (Qiagen, Valencia, Calif.) to remove any residual genomic DNA. RNA concentration was determined before analysis on each of the 10 samples after RNA extraction and each of samples 260/280 ratios were acceptable in the 1.8-2.2 range. Each of the 10 samples then underwent electrophoresis to measure RNA integrity and when all samples met the Affymetrix criteria of being free of DNA with a RIN number of 10, 5 biologic samples of each group were combined and the MRC conducted the Human Genome U133 Plus 2.0 Array/Affymetrix. The microarray data were then normalized by RMA Sketch global normalization in the Affymetrix expression console in order to transform all the arrays to have a common distribution of intensities by removing all technical variation from noisy data before analysis. To quantile normalize two or more distributions to each other, both treated and control groups were set to the average (arithmetical mean) of both distributions. Therefore, the highest value in all cases becomes the mean of the highest values, the second highest value becomes the mean of the second highest values etc. (Bolstad et al. Bioinformatics 2003).

Due to the significant effects that were seen in cell growth/proliferation, cell cycle, membrane potential, and tubulin assay; Affymetrix 2.0 microarray analyses were conducted on both the human breast carcinoma cell line and the human breast epithelial cell in order to see what, if any, effects on gene expression could be measured. Data were normalized with a RMA Sketch Global normalization in an Affymetrix expression console and analyzed with QIAGEN'S Ingenuity Pathway Analysis (IPA) to identify relationships, mechanisms, functions and pathways of relevance in the data. Results of this analysis indicated that the treated group of the carcinoma cell line displayed a significant upregulation in pathways of the Unfolded Protein Response (UPR), Phenylethylamine Degradation 1, tRNA charging and Serine and Glycine Biosynthesis. Strong changes were also displayed in the upstream regulators of TRIB3, PPRC1, ATF4, SCD and GNE. The expression of over 1,000 genes showed a 2-18 fold change after growth in the treated media compared to cells grown in the control media. The significant changes in gene expression that were upregulated in the treated group of the cancerous cells were in the areas of cell survival/death, cell cycle progression, immune modulation and membrane potential. Endoplasmic reticulum (ER) stress leads to a compensatory mechanism in cells referred to as the Unfolded Protein Response (UPR). The UPR is a cellular stress response that is related to ER stress and has been known to be conserved in all mammalian species. The UPR shows significant upregulation in these three arms that are used when the UPR is initiated in order to restore function in the cell: 1) halting protein translation (see PERK-ERN1) 2) degradation of the misfolded proteins (see EDEM2/ERO1-LB) 3) activation of signaling pathways that lead to soliciting the help of molecular chaperones that are involved in protein folding (IRE1, XBP1). (See Logue S E, et al. (2013) J Carcinogene Mutagene.) The UPR also shows an upregulation in the apoptosis or programmed cell death (CHOP/DDIT3/CHAC1) arm. Therefore, the microarray analysis shows significant upregulation in all three arms of the UPR and in the stress response leading to apoptosis of the MDA-MB231 cells. Finally, GADD34 (see below) is a CG3825 gene product from transcript CG3825-RA that binds to PP1 and facilitates translational elongation of specific transcriptional factors that lead to phosphorylation of eIF2-α, thereby terminating global protein translation and inducing apoptosis. (Farook J M, et al. Cell Death and Disease 2013.) There was also a down regulation in many cell cycle progression genes with an upregulation in many genes in the p53 pathway. Significant changes also occurred with an upregulation in the tumor necrosis factors (TNF) as well in the immune responses of several cytokines.

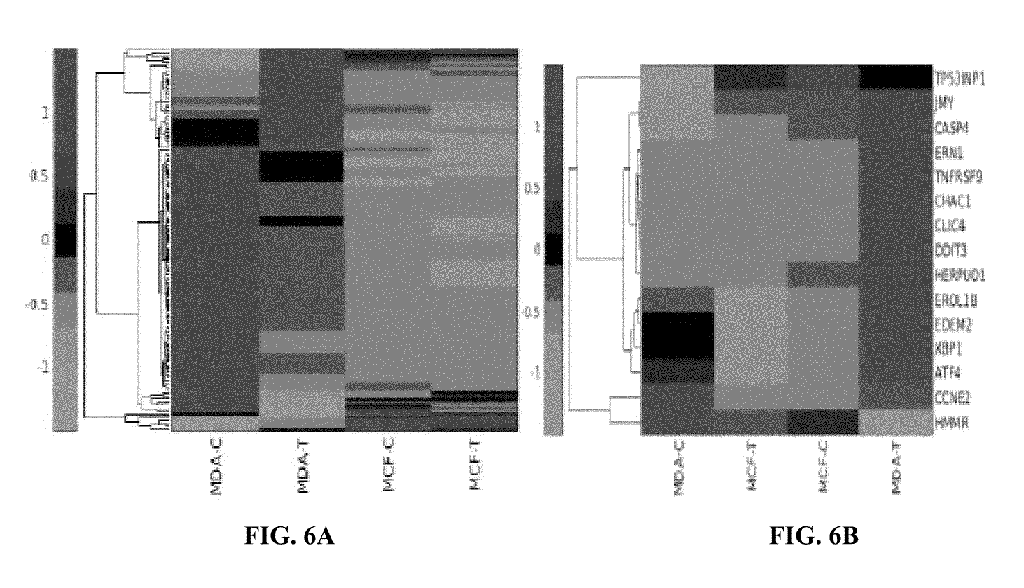

The MCF-10A cell line microarray showed and up regulation in the pathways of: Superpathway of Serine and Glycine Biosynthesis I, Serine Biosynthesis, Role of IL-7A and Granulocyte Adhesions and Diapedesis. While the microarray did show a significant fold increased in the ER Stress and Unfolded Protein Response pathways in the MDA-MB231 cells, it did not show an increased fold change ER Stress or the Unfolded Protein Response in the MCF-10A cells. There was an 8-fold decrease in the CHAC1 expression and this had been shown to correspond to a reversal in ER stress/UPR. (Mungrue I N, et al. Cascade. J. Immunol. 2009.) The MCF10A cells did not show a down regulation of cell cycle progression. There was a greater increase in immune cytokines in the MCF-10A cells when compared to the MDA-MB231 cells. While the MDA-MB231 cells showed a significant fold change in the upregulation of the p53 pathway, there was no up regulation in the tumor necrosis factors or the p53 pathway in the MCF-10A cells. Two heat maps were generated to show both the microarray data and the real time PCR validations (FIGS. 6A & 6B). A list of the gene expressions for both the treated and control groups in both cell lines that were evaluated and validated in order to make biological correlations to our experimental data are listed in TABLE 2.

TABLE 2
Heat Map Affymetrix 2.0 Gene Expression Levels of MDA-MB231
and MCF10A Cells for ER stress/UPR, Immune/TNF, Cell Cycle,
Tumor Targets, Cell Death and Membrane Potential

| Gene | MDA-C | MDA-T | MCF-C | MCF-T |
|---|---|---|---|---|
| ER Stress/UPR | | | | |
| DDIT3 | 92.20325 | 604.7977 | 74.5718 | 38.54762 |
| FBXW10 | 17.46405 | 81.63718 | 12.86469 | 19.93201 |
| ERN1 | 167.6198 | 1715.344 | 180.1033 | 177.2253 |
| CHAC1 | 208.6184 | 3336.654 | 224.6349 | 67.11283 |
| CBLB | 300.1236 | 1641.758 | 293.6763 | 254.1021 |
| HERPUD1 | 1783.141 | 5490.769 | 1948.906 | 1336.873 |
| FAM129A | 885.9388 | 2438.941 | 164.713 | 43.46248 |
| ATF4 | 1885.499 | 3100.368 | 884.2608 | 612.7664 |
| XBP1 | 1657.966 | 3991.217 | 646.7754 | 396.2133 |
| TBC1D3H | 302.5868 | 707.418 | 295.0598 | 210.7293 |
| SEL1L | 1245.15 | 2877.084 | 1030.507 | 742.8039 |
| EDEM2 | 346.4626 | 758.4768 | 246.9249 | 121.0007 |
| EROL1B | 198.7048 | 356.374 | 166.5647 | 130.24 |
| PP1R15A | 621.7018 | 2642.331 | 445.151 | 518.1046 |
| GADD45A | 206.6781 | 855.5336 | 669.77 | 766.9318 |
| TP53INP1 | 92.76067 | 267.6483 | 458.6459 | 322.1881 |
| DRAM1 | 678.105 | 1709.15 | 301.5608 | 301.5608 |
| PRSC1 | 573.9486 | 159.1545 | 89.98862 | 65.02022 |
| GTSE1 | 1697.772 | 476.1245 | 149.0838 | 150.521 |
| JMY | 240.5889 | 1179.91 | 483.9303 | 433.1115 |
| Immune/TNF | | | | |
| IL1A | 50.91498 | 682.6169 | 3773.235 | 6656.809 |
| SPHK1 | 224.5578 | 542.8292 | 74.35 | 84.3 |
| IL8 | 135.5218 | 542.5673 | 285.7594 | 689.56 |
| IL20Rb | 216.931 | 855.3889 | 2492.348 | 2037.954 |
| TNFS4 | 38.17249 | 104.0848 | 31.551 | 29.38311 |
| TNFRSF10 | 1322.594 | 2931.227 | 679.15 | 354.5 |
| IL21R | 88.24291 | 186.0786 | 34.35 | 34.02 |
| TRIB3 | 578.2565 | 2925.584 | 534.79 | 197.3 |
| IL13RA2 | 268.6343 | 714.5335 | 10.6687 | 15.999 |
| OSMR | 1435.055 | 3519.421 | 1430.7 | 1591.7 |
| ABBC3 | 265.6539 | 648.5226 | 1368.9 | 1353.1 |
| TNFRSF9 | 130.5561 | 1854.911 | 45.3355 | 33.309 |
| IFi30 | 3007.301 | 826.8794 | 1181.883 | 1707.668 |
| Cell Cycle | | | | |
| CCNE2 | 1550.488 | 156.7187 | 45.6 | 31.78 |
| HIST1H2BB | 1454.32 | 176.3751 | 43.2 | 68.3 |
| HIST1H2BM | 9383.078 | 904.6331 | 1089.2 | 673.5 |
| HIST1H2AB | 206.6644 | 31.26837 | 16.4 | 15.1 |
| HIST1H3E | 228.3003 | 76.06728 | | |
| HIST1H3H | 182.1673 | 33.49529 | 47.7 | 106.8 |
| HIST1H2AJ | 49.123 | 11.901 | 8.85 | 9.54 |
| HIST1H4B | 450.5416 | 113.0835 | 22.25 | 18.6 |
| HIST1H2AM | 167.8033 | 42.64463 | 23.93 | 16.9 |
| HIST1H3B | 204.3861 | 58.02624 | 11.73 | 6.98 |
| HIST1H4A | 219.4193 | 62.61253 | 21.9 | 15.2 |
| HIST1H2BL | 180.3614 | 54.36535 | 164.2 | 93.9 |
| CASC5 | 2899.345 | 956.1502 | 485.2 | 499.3 |
| HIST2H3D | 139.2912 | 45.06532 | 79.91 | 59.5 |
| HIST2H2AB | 4406.727 | 1618.736 | 1337.66 | 491.5 |
| CDCA7 | 114.252 | 158.884 | 135.68 | 139 |
| CDCA3 | 690.5287 | 128.4345 | 85.279 | 66.4689 |
| MCM10 | 2026.938 | 409.4398 | 146.7 | 95.14 |
| E2F8 | 1701.76 | 344.8467 | 93.04 | 67.344 |
| CENP1 | 1211.37 | 248.7287 | 206.29 | 126.7361 |
| DSCC1 | 1055.73 | 227.4702 | 136.52 | 53.78 |
| CCNA2 | 3625.114 | 813.5735 | 789.6391 | 715.2556 |
| CDKN2C | 529.6627 | 118.9669 | 86.3 | 77.336 |
| LMNB1 | 105.6866 | 23.76363 | 170.453 | 117.7738 |
| CHAF1B | 1302.628 | 306.5765 | 266.99 | 156.226 |
| PCNA | 3565.479 | 859.501 | 708.95 | 627.721 |
| CENPE | 1585.373 | 386.5323 | 384.29 | 280.6339 |
| SPC25 | 973.4363 | 237.5617 | 68.93 | 54.43 |
| CLSPN | 257.1571 | 63.77828 | 45.25 | 46.39 |
| MYBL2 | 663.0643 | 175.7511 | 162.5156 | 195.258 |
| MAD2L1 | 2916.937 | 761.4398 | 261.8003 | 146.9315 |
| CDCA2 | 1287.288 | 343.2617 | 277.99 | 314.51 |
| CDC45 | 2032.203 | 546.5767 | 194.04 | 135.39 |
| DLGAP5 | 3326.605 | 918.2717 | 294.9 | 322.08 |
| SKP2 | 4475.243 | 1275.213 | 1584.21 | 1247.42 |
| GRPR | 462.8514 | 132.1594 | 23.264 | 19.78769 |
| KIF23 | 3014.659 | 1006.878 | 458.2247 | 432.4457 |
| KIF15 | 1047.023 | 351.514 | 210.2767 | 146.1035 |
| CDK1 | 1191.199 | 418.2505 | 302.653 | 219.0567 |
| CDKN3 | 2829.865 | 818.9569 | 558.78 | 441.95 |
| ORC1 | 787.6633 | 228.1306 | 138.7296 | 108.0838 |
| KIF20B | 1491.5 | 485.3235 | 479.7726 | 370.89 |
| CCNB1 | 1460.134 | 437.5147 | 394.2499 | 414.196 |
| PLK1 | 7734.812 | 2320.248 | 2193.42 | 1800.224 |
| CDC20 | 4119.106 | 1241.424 | 1542.313 | 1298.382 |
| TTK | 2063.557 | 623.2984 | 404.7908 | 388.5829 |
| SGOL1 | 2204.15 | 680.1693 | 357.85 | 353.49 |
| CENPK | 965.4617 | 298.0388 | 117.869 | 86.63 |
| CDK15 | 597.6559 | 187.5154 | 25.435 | 22.931 |
| OIP5 | 656.4835 | 210.1888 | 108.08 | 65.89 |
| PRC1 | 3544.327 | 1135.643 | 626.4265 | 457.0201 |
| AURKA | 1372.692 | 441.3315 | 241.9177 | 182.5047 |
| DNA2 | 680.8584 | 220.6961 | 158.5015 | 128.2752 |
| PLK4 | 740.555 | 241.9631 | 118.1282 | 81.45791 |
| MCMY | 1946.111 | 754.5332 | 316.8071 | 318.7342 |
| FOXM1 | 2150.082 | 834.0797 | 453.6553 | 534.8372 |
| CENPO | 1545.834 | 528.9465 | 173.4009 | 134.9089 |
| CDC25A | 906.8274 | 355.5253 | 81.97407 | 73.47563 |
| KIF4A | 1226.718 | 483.1996 | 285.4267 | 325.8651 |
| BORA | 833.334 | 333.975 | 177.7702 | 161.6121 |
| PRIM1 | 1933.796 | 639.5529 | 289.6865 | 144.092 |
| KIF23 | 3014.659 | 1006.878 | 458.2247 | 432.4457 |
| BUB1 | 3482.412 | 1171.759 | 535.3447 | 538.2644 |
| NUF2 | 1068.651 | 362.4769 | 158.0058 | 117.314 |
| CENPA | 1604.808 | 623.0319 | 259.9626 | 173.262 |
| NEK2 | 1471.66 | 508.0363 | 798.9699 | 809.4927 |
| CDC25C | 559.6721 | 194.3788 | 150.2855 | 97.89624 |
| CDK15 | 597.6559 | 187.5154 | 25.436 | 22.93094 |
| CIT | 708.1124 | 251.2761 | 290.004 | 225.4576 |
| CCNF | 509.6929 | 181.4399 | 143.653 | 115.1559 |
| CDCA8 | 2397.439 | 856.6898 | 532.59 | 448.22 |
| ASF1B | 3257.62 | 1238.113 | 417.605 | 193.1282 |
| CENPL | 407.7961 | 156.0273 | 124.78 | 103.6942 |
| CCNA1 | 1235.768 | 475.7696 | 132.0476 | 199.9614 |
| CENPM | 559.2139 | 212.9774 | 116.38 | 91.68 |
| KIF11 | 3238.945 | 939.4666 | 708.69 | 495.69 |
| CDC45 | 2032.203 | 546.5767 | 194.04 | 135.38 |
| CENPF | 1518.461 | 633.5304 | 1067.66 | 1123.12 |
| Cell Death | | | | |
| CASP4 | 78.27916 | 167.4708 | 99.7621 | 93.61928 |
| CASP6 | 315.9396 | 153.2165 | 110.2821 | 103.9024 |
| UNC5B | 165.5258 | 1028.606 | 765.4645 | 432.6196 |
| Tumor Targets | | | | |
| CARS | 410.5192 | 1051.12 | 212.4383 | 107.2973 |
| BEX2 | 54.2207 | 128.0799 | 39.484 | 39.164 |
| JUNB | 700.5781 | 1589.828 | 1304.435 | 1068.991 |
| HMMR | 681.7925 | 183.141 | 504.2788 | 362.3813 |
| PTTG1 | 267.0026 | 72.87782 | 45.128 | 40.01671 |
| FANCA | 907.874 | 292.4976 | 196.06 | 128.35 |
| TGFB2 | 2404.895 | 897.6522 | 212.696 | 258.4106 |
| FAM83D | 2544.584 | 698.6316 | 700.476 | 661.334 |
| KIFC1 | 1314.23 | 428.6075 | 179.392 | 145.62 |
| E2F1 | 2050.937 | 671.5983 | 108.8849 | 92.29729 |
| Membrane | | | | |
| Potential | | | | |
| CLIC4 | 2043.479 | 5258.273 | 2021.464 | 1791.211 |
| CLIC2 | 57.95258 | 117.7829 | 345.69 | 274.86 |

### Example 5

RT-PCR analyses: In order to validate some of the significant genomic effects seen in the microarray analysis on the MDA-MB231 cells, we conducted Real-Time PCR using LC 480 and UPL probes. Ten genes were chosen: (1) CHaC glutathione-specific gamma-glutamylcyclotransferase 1 (CHAC1), (2) Endoplasmic Reticulum to Nucleus Signaling 1 (ERN1), (3) Homocysteine-Inducible, Endoplasmic Reticulum Stress-Inducible, Ubiquitin-Like Domain Member 1 (HERPUD1), (4) Tumor Necrosis Factor Receptor Family 9 (TNFRSF9), (5) Junction-mediating and regulatory protein of p53 (JMY), (6) Cyclin E2 (CCNE2), (7) Hyaluronan-mediated motility receptor (HMMR), (8) DNA-Damage-Inducible Transcript 3 (DDIT3), (9) Caspase 4 (CASP4), (10) Chloride Intracellular Channel Protein 4 (CLIC4) and RIBOPROTS19 (housekeeping). The primers were designed using universal probe library. Transcriptor First Strand cDNA Synthesis Kit (Roche Cat. No. 04 379 012 001) was used to make cDNA with the original microarray samples. Then the following reagents were added to the wells in the appropriate measurements according to the protocol in order to make 8 mcl of this master mix for each well used in the 5-dilution factors (in triplicate) of the cDNA: universal library probe (UPL probe, Roche) at 10 uM, LC480 master mix (2× concentration, Roche), mixed left and right primers at 10 uM each in DNase, RNase & Protease-free water (Corning cellgro, #46-000 Cl), and nuclease free water. The five dilutions of cDNA were: undiluted, 1:10, 1:100, 1:1,000, 1:10,000. The 8 mcl of the master mix and 2 mcl of the cDNA were added to wells of a 96 well plate. The plates were centrifuged and activated for 5 minutes at 95 degrees. Then there was an amplification of 45 thermal cycles using 50° C. for 2 minutes (binding), and 95° C. for 15 seconds (denaturation) and 60° C. for 1 minute (polymerization) and the amplicons were then plotted for validation.

Once all primers were tested for appropriate efficiency of 1.8-2.1 based on conformity of the standard deviations of all of the dilutions, cDNA was made from 5 biological control samples of the MDA-MB231 cells and 5 biological treated samples of the MDA-MB231 cells using the Transcriptor First Strand cDNA Synthesis Kit (Roche, Cat. No. 04 379 012 001). The protocol was followed using: Transcriptor Reverse Transcriptase, Transcriptor RT Reaction Buffer (5×), Protector RNase Inhibitor, Deoxynucleotide Mix, Anchored-oligo (dT) Primer, Random Hexamer Primer, Control and Treated RNA, Primer Mix PBGD and PCR-grade Water.

The RNA and all the above listed reagents were then placed in a thermal block LightCycler 480 instrument with a heated lid and run through the 60 minute cycled experimental program for denaturation, amplification, melting and cooling. The cDNA was placed in a −80° C. freezer until use for real-time PCR.

Real-time PCR for each gene was run in triplicate for each of the 5 control and 5 treated biological samples. Relative quantification was used to analyze the changes in the gene expression in the samples relative to the reference sample that was analyzed from each of the biological replicates. Amplification curves were noted on all the gene samples (not shown) and each sample was run in triplicate (not shown).

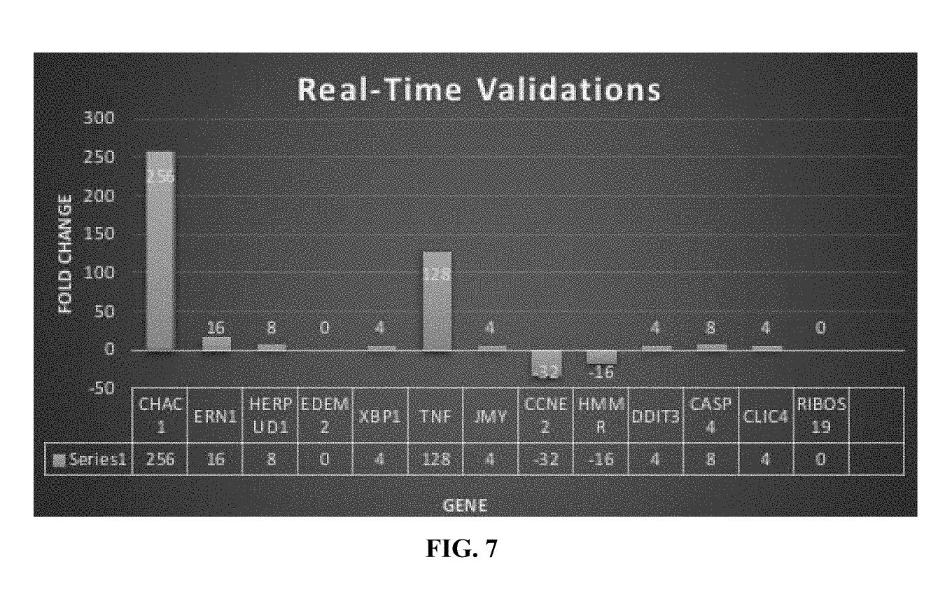

The ER Stress/UPR pathway genes of: ERN1, HERPUD1, XBP1, DDIT3 and CHAC1 all showed a significant increase in gene expression in the treated MDA-MB231 cells. The ER Stress/UPR showed no up regulation in the MCF-10A by validation of DDIT3. The CHAC1 gene in treated MDA-MB231 cells showed a 256-fold increase which shows a strong response in the apoptotic arm of the UPR after 3 days in the treated media, but was significantly down regulated in the MCF-10A cells. (Mungrue I N, et al. J. Immunol. 2009.) The TNFRSF9 gene showed a 128-fold increase in a player in the TNF/TRAIL pathway. TNFRSF9 has been shown to be expressed in activated T cells (CD8 & CD4), dendritic cells, natural killer cells, granulocytes and blood vessel inflammation. (Vinay D S, Kwon, B S. BMB Reports. 2014.) It has also been shown to stop tumors in mice. (Schwartz H. Journal of Leukocyte Biology. 2005.) The Junction Mediating Protein (JMY) is a p53 cofactor that codes a tumor suppressor protein and is up regulated 4-fold in our validations. It is believed to be a key transcriptional regulator and controls DNA repair, cell cycle progression, angiogenesis and apoptosis. (Coutts A S, LaThangue N. Biochem. Soc. Symp. 2006.) The loss of p53 function is thought to be a contributing factor in the majority of cancer cases. Caspase 4 (CASP4) is the caspase that is linked to ER Stress/UPR and it is up regulated 8-fold. (Kim S J, et al. Hum Mol Genet. 2006.) Cyclin E2 (CCNE2) is down regulated 32-fold and has been shown to be elevated in tumor-derived cells and plays a role in the G1/S transition in the cell cycle. (Gudas, J M, et al. Molecular and Cellular Biology 1999.) Hyaluronan-mediated motility receptor (HMMR) interacts with BRCA land other proteins to control key aspects of cell polarity and cell division and may hold answers to how to treat women with BRCA1 and BRCA2 mutations as its expression and overexpression has been linked to ras transformation, tumor progression and metastasis. (Maxwell C, et al. Mol Biol Cell. 2003.) HMMR is down regulated 16-fold on our validations. Chloride Intracellular Channel 4 (CLIC4) is a group of proteins who regulate cell membrane potential, transepithelial support, maintain pH and cell volume. Under expression or reduced CLIC4 alters the redox state of tumor cells and enhances tumor development. (Suh K S, et al. Carcinogenesis 2012.) It is upregulated 4-fold in our gene validations (See FIG. 7).

The CLIC4 gene is known to participate in membrane potential regulation and is a known tumor suppressor. (Suh K S, et al. Carcinogenesis 2012.) This gene is upregulated 4-fold in the treated MDA-MB231 cells in the RT-PCR validation but was not upregulated in the noncancerous cells. This genomic change in expression in a gene in the cancerous cells that controls membrane potential regulation corresponds to the membrane potential assay testing. This suggests the treated media shows an effect in the regulation of membrane potential in cancerous cells who are known to possess a depolarized membrane when compared to a noncancerous cell.

The microarray and RT-PCR validations on the MDA-MB231 cells showed the unfolded protein response (UPR) as the top pathway affected significantly by the CETS unit and this pathway was not upregulated in the microarray and the RT-PCR of the MCF-10A cells. The UPR is a series of signaling events that occur due to intracellular stress from misfolded proteins in the lumen of the endoplasmic reticulum (ER). Certain pathologic stimuli can cause an interruption in the protein folding process and these include but are not limited to: calcium depletion, altered glycosylation, nutrient deprivation, oxidative stress, DNA damage or energy fluctuations. (Li X, et al. J Hematol Oncol. 2011.) While no significant increase in protein synthesis was found with the BCA protein assay of human breast carcinoma, data showed a 2.5 fold increase in mRNA between the treated and control groups. The increase in mRNA indicates a possible increase in transcriptional affects may be initially occurring prior to the up regulation of ER stress/UPR pathways. The cell size appears to increase initially in our cell size data and the cells then begin to shrink which could suggest the halting of protein synthesis and the ER degradation/protein degradation that occurs with the UPR survival response. UPR has been shown to be activated by increased activation of ATF4 and increased transcription of its target C/EBP homologous protein (CHOP), which is a pro-apoptotic factor. (Nogalska A, et al. J Neuropathol Exp. Neurol. 2015.) ATF4 is a strong upstream regulator in our microarray analysis. The known causes of UPR are energy fluctuations. This suggests a possible connection between the significant changes in the membrane potential of the cancerous cell lines and the upregulation of the UPR. The treated media did not illicit a growth inhibition in the noncancerous cells, which shows that growth arrest from UPR is likely not occurring in the noncancerous cells and also lends to the thought that other known causes of ER stress related to nutrient or calcium deprivation from the media are most probably not originating factors in the cancerous cell lines. The RT-PCR validated that ER Stress/UPR gene expression was not up regulated and this corresponds to the experimental findings with the MCF-10A cells. The cell growth studies found cancerous cell lines significantly slowed mitosis and halted their cell cycles. In ER stress there is a struggle to balance adaptation and alarm/death. UPR is often highly activated in cancer cell to promote survival. In the RT-PCR validations, ERN1 is upregulated 14-fold, which could suggest one mechanism for cell cycle arrest and DDIT3 (CHOP) is upregulated 4-fold, while CHAC1, which is downstream from CHOP is strongly upregulated 256-fold and could indicate that these cancer cells are headed down the apoptosis arm of the UPR after 3 days of exposure to the treated media. (Brewer J W, Diehl J A. Proc Natl Acad Sci USA 2000.) CHAC1 is the pro-apoptotic component of the unfolded protein response pathway that mediates the pro-apoptotic effects of the ATF4-ATF3-DDIT3-CHOP cascade. (Mungrue I N, et al. J. Immunol. 2009.) Caspase 4 was significantly upregulated 4-fold as well in the RT-PCR and this is the caspase that is linked to the UPR and could suggest a possible beginning to a cell death. (Hitomi J, et al. J Cell Biol. 2004.) An unsuccessful UPR can also be caused by an increase in TNF, which was also significantly upregulated 126-fold in the RT-PCR and operates down the IRE1-TRAF2-JNK pathway in ER stress. (Colbert R A, et al. Immunol Rev. 2010.) TRIB3 was shown to be a significant upstream regulator in our microarray, which is a known sensitizer of cells to TNF and TRAIL induced apoptosis. (Fang N, et al. Metabolism 2014.)

In our microarray, TP53INP1 is significantly upregulated gene that is involved in making a protein that has anti-proliferative and pro-apoptotic properties and acts as a regulator of transcription and autophagy. This gene plays a major role in the p53/TP53 oxidative stress response and was also validated with RT-PCR as a 4-fold increase with JMY. It is possible that ER stress/degradation and anything that leads to the UPR could nonspecifically halt DNA replication leading to p53 activation and apoptosis. (Stavridi E, Halazonetis T. Genes & Dev. 2004; Yoshida K, et al. J. Biol. Chem. 2006.) TP53INP1 can also reduce cell migration by regulating the expression of SPARC. (Thomas S L. Brain Pathol. 2015.)

The microarray found that TNFRSF9 was also found to be significantly upregulated downstream from the also significantly affected ER transmembrane protein: Inositol Requiring 1 (IRE1). Prolonged ER stress has been shown to activate the pro-apoptotic IRE1-TRAF2-JNK pathway. Tumor necrosis factor receptor superfamily, member 9 is a member of the TNF receptor superfamily that is implicated in the survival and development of T cells. It is a significant player in the 4-1BB Signaling in T lymphocytes pathway, which is known for eradication of established tumors, enhancing integrin-mediated cell adhesion and increasing T cell cytolytic potential. TNFRSF9 is also linked to the Death Receptor Signaling and NF-KB pathways. (Schwartz H, Tuckwell J, Lotz M. Gene 1993.) While tumor necrosis factors are not playing a role in these in vitro experiments, they could be a strong player in a future in vivo model. Also, CLIC4 codes for a diverse group of proteins that regulates cellular processes, such as stabilization of cell membrane potential. CLIC4 has been shown to participate in suppression of tumor growth and the absence of decreased levels of CLIC4 has been found to contribute to TGF-β resistance and enhances tumor development. (Suh K S, et al. Carcinogenesis 2012.) Membrane potential assay results corroborate this finding (data not shown). These significant changes of CHOP, CHAC1, JMY/P53, and CASP4 in the microarray/RT-PCR, Annexin V fluorescent microscopy and the noted microscopic effects of nuclear fragmentation (data not shown), blebbing and decrease in cell size provide evidence of a possible apoptosis or other form of undetermined cell death may be occurring in these treated human breast carcinoma. Interestingly, the MCF10A cell line did not show these experimental or genomic effects and show this could be a possible side-effect free adjunct therapy for cancer patients.

The microarray and RT-PCR validations show the cancer cells heading down several pro-apoptotic pathways related to ER stress/degradation, as well as the p53 oncogene activation pathway while these validations also show this effect is not occurring in the noncancerous cells. The mechanism of triggering p53 signaling during ER stress induced apoptosis is currently unknown but may possibly be associated with a hyperpolarized membrane according to both our experimental findings and the genomic analyses. Two of the top pathways delineated in our microarray according to our Ingenuity analysis were also shown to be serine biosynthesis and the super pathways of serine and glycine biosynthesis. The tumor suppressor p53 has been classically known to regulate DNA repair, cell-cycle arrest and apoptosis. In the process, these actions will upregulate metabolic targets and thereby upregulate these pathways of biosynthesis. (Tavana O, Gu W. Cell Metabolism 2013.) The upregulation of serine and glycine biosynthesis is an essential reversal of how cancer cells can usually reprogram their metabolism by shifting from oxidative phosphorylation to aerobic glycolysis (Warburg effect) in order to achieve unchecked growth. (Ward P S, Thompson C B. Cancer Cell 2012.) The amplification of metabolic enzymes has been found in breast, liver, prostate and melanoma cancers due to the identified amplification of the gene encoding phosphoglycerate dehydrogenase (PHGDH) leading to increase flux through the serine/glycine pathway. (Locasale J, et al. Nature Genetics 2011.) PHGDH increase has been linked to increased proliferation of cancerous cell lines. (Id.) Our data suggest an increase in PHGDH and the serine/glycine super-pathways can also be possibly linked to decreased proliferation in cancerous cells lines under the right conditions, while conversely, this pathway was also upregulated in our noncancerous cell line, which did not show a halt in cell cycle or cell growth. In recent years, the study of metabolism has returned to the forefront of cancer research. Data now support that altered metabolism can possibly result from the reprogramming by altered oncogenes and tumor suppressors. Our data suggest that restoring the altered oncogene and tumor suppressor functions could return the cell to a normal metabolism. Altered metabolism as well as an altered immune response should continue to be considered two major hallmarks of cancer that is studied in future research.

The cancer cells appear to have increased anabolic function with the upregulation of the serine/glycine super pathways (nucleotide formation), increased catabolic function with the upregulation of phenylethylamine degradation pathway (carbon and nitrogen source) and reprogramming of the UPR and p53 genomic apoptotic mechanisms. There are many forms of cell death that include but are not limited to apoptosis, entosis, mitotic catastrophe, necrosis, necroptosis, excitotoxicity, autophagic cell death, and pyrotosis. (Kromer G, Galluzze L, Melino G. et al. Cell Death Diff. 2009.) Dying cells are engaged in a process that is reversible until a “point of no return” is passed. (Id.) The MDA-MB231 cells appear to be headed down a cell death pathway, (Suh K S, et al. Carcinogenesis 2012) while the MCF-10A cells do not. The cancerous cells that appear to survive the exposure to the treated media also appear to display different characteristics of slower cell growth when placed back in standard/non-treated media (data not shown). This slower cell growth could suggest some form of change in cell metabolism/function. Many other associated gene expressions that are linked to control of tumorigenesis, tumor development, cell migration and cell differentiation have been shown significant in our microarray and warrant more research. The upregulation of IL1A (IL10) and TNF also have been linked to an unsuccessful UPR and could also lead to research in the autoimmune response to disease. There are also strong down regulatory changes that are linked to cell cycle check points as well as cell cycle progression. Hyperpolarization of the plasma membrane could in theory be a reprogramming of a cancer cell to behave like a normal cell that has lost its ability to function as a beneficial member to the organism; whereas a cell cycle arrest and eventual cell death cascade is initiated. Hyperpolarization of the noncancerous cell line does not appear to similarly affect these same pathways. There appears to be a slight down regulation of the ER Stress/UPR pathway, which could show promise for other chronic diseases such as neuro-generative diseases, organ fibrosis and diabetes that have also been linked to an aberrant UPR. There is a significant differential effect with regards to ER Stress/UPR and many other metabolic functions of the cell with the CETS treated water when one compares the cancerous cell versus noncancerous cell line responses.

Throughout this document, various references are mentioned. All such references are incorporated herein by reference as though individually incorporated by reference, to the extent each reference is at least partially not inconsistent with the disclosure in the present application (for example, a reference that is partially inconsistent is incorporated by reference except for the partially inconsistent portion of the reference), including the references set forth in the following list:

### REFERENCES

Bolstad B, Irizarry R, Astrand M, Speed T. A comparison of normalization methods for high intensity oligonucleotide array data based on variance and bias. Bioinformatics 2003; 19 (2): 185-193.
Brewer J W, Diehl J A. PERK mediates cell-cycle exit during mammalian unfolded protein response. Proc Natl Acad Sci USA 2000; 97 (23): 12625-30.
Charman, R. A. (1996). Electrotherapy. London, U K: W.B. Saunders Company Ltd.
Chen, Kevin W. Inhibitory Effects Of Bio-Energy Therapies On Cancer Growth, World Science and Technology/Modernization of Traditional Chinese Medicine and Materia Medica, Vol. 10 No. 4, 2008.
Chen, Y. O. D. U. D. o. B. S. (2012). Nano-and micro-second electrical pulsing of B16-F10 mouse melanoma cells: plasma membrane and sub-cellular organelle changes. Available from http://worldcat.org/z-wcorg/database.
Chernet, B. T., & Levin, M. (2013). Transmembrane voltage potential is an essential cellular parameter for the detection and control of tumor development in a Xenopus model. Dis Model Mech, 6(3), 595-607. doi: 10.1242/dmm.010835.
Cone, C. D., Jr. (1970). Variation of the transmembrane potential level as a basic mechanism of mitosis control. Oncology, 24(6), 438-470.
Colbert R A, Delay M L, Klenk E I, Layh-Schmitt G. From HLA-B27 to spondyloarthritis: a journey through the ER. Immunol Rev. 2010; 233(1):181-202.
Coutts A S, LaThangue N. The P53 response during DNA damage: impact of transcriptional cofactors. Biochem. Soc. Symp. 2006; (73): 181-9.
Davis, B. (2004). Structured Water is Changing Models. The Scientist. Retrieved Mar. 21, 2014.
Demming, A. (2013). Nanotechnological selection. Nanotechnology, 24(2), 020201. doi: 10.1088/09574484/24/2/020201.
Fang N, Zhang W, Xu S, Lin H, Wang Z, Liu H, Fang Q, Li C, Peng L, Lou J. TRIB3 alters endoplasmic reticulum stress-induced B-cell apoptosis via the NF-kB pathway. Metabolism 2014; 63 (6): 822-30.
Farook J M, Shields J, Tawfik A, Markand S, Sen T, Smith S B, Brann D, Dhandapani K, Sen N. GADD34 induces cell death through inactivation of Akt following traumatic brain injury. Cell Death and Disease 2013; 4:e754.
Funk, R. H., Monsees, T., & Ozkucur, N. (2009). Electromagnetic effects—From cell biology to medicine. Prog Histochem Cytochem, 43(4), 177-264. doi: 10.1016/j.proghi.2008.07.001.
Gregory et al., The Water Dipole Moment in Water Clusters, Science 275,814 (1997).
Gudas, J M, Payton, M, Thukral S, Chen E, Bass Michael, Robinson M O, Coats S. Cyclin E2, a novel cyclin that binds CDK2 and is aberrantly expressed in human cancers. Molecular and Cellular Biology 19 (1): 612-22 (1999).
Habal M. Effect of applied dc currents on experimental tumor growth in rats. Journal of Biomedical Materials Research; 1980. 14(6), 789-801.
Hitomi J, Katayama T, Eguchi Y, Kudo T, Taniguchi M, Koyama Y, Manabe T, Yamagishi S, Bando Y, Imaizumi K, Tsujimoto Y, Tohyama M. Involvement of caspase4 in endoplasmic reticulum stress-induced apoptosis and Abeta-induced cell death. J Cell Biol. 2004; 165 (3): 347-56.
Hu & Verkman, Increased Migration And Metastatic Potential Of Tumor Cells Expressing Aquaporin Water Channels, The FASEB Journal, 2006.
Huang et al., The influence of electric field and confinement on cell motility, PLoS One, March 2013, Volume 8, Issue 3, e59447.
Kalaycio, M. (2014). Guidelines, predictive models needed to improve understanding of secondary malignancies. HemOnc today, 15(8), 1.
Kim S J, Zhang A, Hitomi E, Lee Y C, Mukherjee A B. Endoplasmic Reticulum stress-induced caspase-4 activation mediates apoptosis and neurodegeneration in INCL. Hum Mol Genet. 2006; 15 (11): 1826-34.
Kromer G, Galluzze L, Melino G. et al. Classification of cell death, recommendations of the Nomenclature Committee on Cell Death. Cell Death Diff 2009; 16 (1): 3-11.
Levin, M. (2007). Large-scale biophysics: ion flows and regeneration. Trends Cell Biol, 17(6), 261-270. doi: 10.1016/j.tcb.2007.04.007.
Li X, Zhang K, Li Z. Unfolded protein response in cancer: the physician's perspective. J Hematol Oncol. 2011; 23 (4).
Lobikin, M., Chernet, B., Lobo, D., & Levin, M. (2012). Resting potential, oncogene-induced tumorigenesis, and metastasis: the bioelectric basis of cancer in vivo. Phys Biol, 9(6), 065002. doi: 10.1088/1478-3975/9/6/065002.
Locasale J, Grassian A, Melman T, Lyssiotis C, Mattaini K. Phosphoglycerate dehydrogenase diverts glycolytic flux and contributes to oncogenesis. Nature Genetics 2011: 43: 869-874.
Logue S E, Gorman A M, Cleary P, Keogh N, Samali A. (2013). Current Concepts in E R Stress-Induced Apoptosis. J Carcinogene Mutagene S6:002.
Marsh, R. (2001). A Preliminary Report on the New BEFE: Bioelectric Field Enhancement. EXPLORE, 10(6).
Maxwell C, Keats J, Crainie M, Xuejun S, Yen T, Shibuya E, Hendzel M, Chan G, Pilarski L. Mol Biol Cell. 2003; 14 (6): 2262-2276.
Mayer, Kevin. Opening Of Water Channel Coincident With Receptor Activation, GEN News Highlights: Sep. 10, 2014.
Mijovic, Z., Kostov, M., Mihailovic, D., Zivkovic, N., Stojanovic, M., & Zdravkovic, M. (2013). Correlation of nuclear morphometry of primary melanoma of the skin with clinicopathological parameters and expression of tumor suppressor proteins (p53 and p16(INK4a)) and bcl-2 oncoprotein. J buon, 18(2), 471-476.
Movaffaghi & Farsi, biofield therapies: biophysical basis and biological regulations? Complementary Therapies in Clinical Practice 15, (2009) 35-37.
Mungrue I N, Pagnon J, Kohannim O, Gargalovic P S, Lusis, A J. CHAC1/MGC4504 is a Novel Proapoptotic Component of the Unfolded Protein Response, Downstream of the ATF4-ATF3-CHOP Cascade. J. Immunol. 2009; 182 (1): 466-476.
Mycielska, M. E., & Djamgoz, M. B. (2004). Cellular mechanisms of direct-current electric field effects: galvanotaxis and metastatic disease. J Cell Sci, 117 (Pt 9), 1631-1639. doi: 10.1242/jcs.01125.
Nogalska A, D'Agostino C, Engel W K, Cacciottolo M, Asada S, Mori K, Askanas V. Activation of the Unfolded Protein Response in Sporadic Inclusion-Body Myositis but Not in Hereditary GNE Inclusion-Body Myopathy; J Neuropathol Exp. Neurol. 2015; 74 (6): 538-46.
Ohnishi et al., Growth Inhibition Of Cultures Human Liver Carcinoma Cells By Ki-Energy (Life-Energy): Scientific Evidence For Ki-Effects On Cancer Cells, eCAM 2005; 2(3)387-393.
Ojeda-May & Garcia, Electric Field-Driven Disruption Of A Native Beta-Sheet Protein Conformation And Generation Of A Helix-Structure, Biophysical Journal, Volume 99 July 2010, 595-599.
Panagopoulos, et al., Mechanism For Action Of Electromagnetic Fields On Cells, Biochemical And Biophysical Research Communications, 298 (2002) 95-102.
Romanel et al., Transcriptional Regulation Is A Major Controller Of Cell Cycle Transition Dynamics, PLoS One, 2012.
Schwartz H. Biological activities of reverse signal transduction through CD137 ligand. Journal of Leukocyte Biology. 2005; 77 (3): 281-286.
Schwartz H, Tuckwell J, Lotz M. A receptor induced by lymphocyte activation (ILA): a new member of the human nerve-growth-factor/tumor-necrosis-factor receptor family. Gene 1993. 134:295-298.
Stavridi E, Halazonetis T. P53 and stress in the ER. Genes & Dev. 2004; 18: 241-244.
Suh K S, Malik M, Shukla A, Ryscavage A, Wright L, Jividen K, Crutchley J M, Dumont R A, Fernandez-Salas E, Webster J D, Simpson R M, Yuspa S H. CLIC4 is a tumor suppressor for cutaneous squamous cell cancer. Carcinogenesis 2012; 33 (5); 986-95.
Sundelacruz, S., Levin, M., & Kaplan, D. L. (2009). Role of membrane potential in the regulation of cell proliferation and differentiation. Stem Cell Rev, 5(3), 231-246. doi: 10.1007/s12015-009-9080-2.
Tavana O, Gu W. The Hunger Games: p53 Regulates Metabolism upon Serine Starvation. Cell Metabolism 2013; 17 (2): 159-161.
Thomas S L. Loss of SPARC in p53-null Astrocytes Promotes Macrophage Activation and Phagocytosis Resulting in Decreased Tumor Size and Tumor Cell Survival. Brain Pathol. 2015; 25(4): 391-400.
Tseng, A., & Levin, M. (2013). Cracking the bioelectric code: Probing endogenous ionic controls of pattern formation. Commun Integr Biol. 6(1), e22595. doi: 10.4161/cib.22595.
Vinay D S, Kwon, B S. 4-1BB (CD137), an inducible costimulatory receptor, as a specific target for cancer therapy. BMB Reports. 2014; 47 (3): 122-129.
Wahl, A. (2005). A short history of electrochemistry. Galvanotechtnik, 96(8), 1820-1828.
Walker, S. (1998). Hydroponic Treatment Trial. from http://www.genergyspa.com/?contentID=126.
Ward P S, Thompson C B. Metabolic Reprogramming: a cancer hallmark even warburg did not anticipate. Cancer Cell 2012; 21: 297-36.
Wild, C. (2014). WHO projects ‘alarming rise’ in cancer worldwide. HemOnc today, 15(6), 6.
Woltz, P. S. a. S. (1990). Successful Biological Control of Cancer: By Combat Against the Causes. Gesamtherstellung: Neuwieder Verlagsgesellschaft mbH.
Yang, M., & Brackenbury, W. J. (2013). Membrane potential and cancer progression. Front Physiol, 4, 185. doi: 10.3389/fphys.2013.00185.
Yoshida K, Liu H, Miki Y. Protein kinase C delta regulates Ser46 phosphorylation of p53 tumor suppressor in the apoptotic response to DNA damage. J. Biol. Chem. 2006; 281: 5734-5740.
Zhou, S.-A., & Uesaka, M. (2006). Bioelectrodynamics in living organisms. International journal of engineering science, 44(1), 67-92.
Zimmerman, J. W., Pennison, M. J., Brezovich, I., Yi, N., Yang, C. T., Ramaker, R., Pasche, B. (2012). Cancer cell proliferation is inhibited by specific modulation frequencies. Br J Cancer, 106(2), 307313. doi: 10.1038/bjc.2011.523.

We claim:

1. A method of the therapeutic treatment of a disorder in a subject, comprising:
applying an electric field that inhibits the growth of the cancer cells, but leaves normal cells substantially unharmed, wherein the subject or a portion of the subject is immersed in a therapeutically effective amount of a composition exposed to the electric field.

2. The method of claim 1, wherein the composition is in solution.

3. The method of claim 1, wherein the composition is subjected to an electric field of between about 100 mV and 900 mV and up to 2.5 amperes of direct current.

4. The method of claim 1, wherein the composition is subjected to an electric field of 130 mV and 2.5 amperes of direct current.

5. The method of claim 1, wherein the composition is subjected to an electric field using a cellular energy transfer system (CETS).

6. The method of claim 1, wherein the composition comprises an electro-activated solution.

7. The method of claim 2, wherein the solution is sodium chloride solution at concentration of about 2 mM to about 5 mM.

8. The method of claim 1, wherein the composition was subjected to an electric field for a duration of up to about 35 minutes.

9. The method of claim 1, wherein the therapeutic treatment results in inhibition of cancer cell growth.

10. The method of claim 1, wherein the electric field is generated using a series of spaced rings.

11. The method of claim 1, wherein the disorder is cancer.

12. A method for the therapeutic treatment of a disorder comprising: subjecting a therapeutic agent to an electric field; and applying a therapeutically effective amount of the therapeutic agent to a subject in need of treatment therefrom, to thereby limit the occurrence of cancer or treat cancer in the subject.

13. The method of claim 12, wherein subjecting a therapeutic solution to an electric field comprises subjecting the therapeutic agent to an electric field of between about 100 mV and about 900 mV and up to about 2.5 amperes of direct current.

14. The method of claim 13, wherein subjecting a therapeutic agent to an electric field comprises subjecting the therapeutic agent to an electric field of 130 mV and 2.5 amperes of direct current.

15. The method of claim 12, wherein the electric field is generated using a cellular energy transfer system (CETS).

16. The method of claim 12, wherein subjecting a therapeutic agent to an electric field comprises subjecting a therapeutic agent to an electric field for a duration of up to about 35 minutes.

17. The method of claim 12, wherein the therapeutic treatment results in inhibition of growth of cancer cells.

18. The method of claim 12, wherein subjecting a therapeutic agent to an electric field comprises generating an electric field using a series of spaced rings.

19. A composition prepared by a process comprising the steps of: providing a composition comprising a conductive agent, provide an electric field of between about 100 mV and about 900 mV and up to about 2.5 amperes of direct current, and subject the composition under the electric field for a duration of up to 35 minutes.

20. The compositing of claim 19, wherein the composition is an electro-activated solution.
